(19) United States
(12) Patent Application Publication
(10) Pub. No.: US 2004/0254368 A1
(43) Pub. Date: Dec. 16, 2004

(54) Production of aldoses

(75) Inventors: Kazuhiko Togashi, Sodegaura-City (JP); Hideki Umetani, Sodegaura-City (JP); Tomoyuki Ando, Sodegaura-City (JP); Hiroki Ishibashi, Omuta-City (JP); Yasushi Fukuiri, Omuta-City (JP); Atsushi Sakuma, Omuta-City (JP)

Correspondence Address: BURNS DOANE SWECKER & MATHIS L L P, POST OFFICE BOX 1404, ALEXANDRIA, VA, 22313-1404, US

(73) Assignee: Mitsui Chemicals, Inc., Tokyo (JP)

(21) Appl. No.: 10/864,426

(22) Filed: Jun. 10, 2004

(30) Foreign Application Priority Data
Jun. 12, 2003 (JP) 2003-167380

(51) Int. Cl. C07H 1/00

(52) U.S. Cl. 536/124

(57) ABSTRACT

An aldose having n-1 carbon atoms is produced from an aldonic acid having n carbon atoms using hypochlorous acid or a hypochlorite in a high yield at low cost with safety, by treating the reaction mixture with a compound having reactivity with the hypochlorous acid or hypochlorite higher than that with the produced aldose.

### BACKGROUND OF THE INVENTION

1. Field of the Invention

The present invention relates to a process for producing an aldose. More specifically, it relates to a process for producing an aldose having n-1 carbon atoms from an aldonic acid having n carbon atoms using hypochlorous acid or a hypochlorite.

2. Description of the Related Art

Aldonic acids and other sugar-related compounds are degraded by various processes. Typical examples of processes for producing an aldose having n-1 carbon atoms from an aldonic acid having n carbon atoms are as mentioned below.

(1) A process of preparing arabinose from calcium gluconate by decarboxylation with hydrogen peroxide in the presence of an iron catalyst to yield arabinose (Berichte, 31, 1573 (1898); and J. Am. Chem. Soc., 72, 4546 (1950));

(2) a process of preparing arabinose from gluconic acid by decarboxylation by action of electrolytic oxidation (Annual Report of the Noguchi Institute, 25, 26 (1982));

(3) a process of producing arabinose from gluconolactone via gluconic acid by decarboxylation with cerium sulfate (J. Chem. Soc., Perkin Trans. II, 685 (1977));

(4) a process of preparing arabinose from gluconolactone via sodium gluconate by decarboxylation with sodium hypochlorite under acidic conditions (J. Am. Chem. Soc., 81, 5190 (1959)); and

(5) modified processes of the process (4) (Japanese Unexamined Patent Application Publications No. 55-164699 and No. 8-231448).

However, the process (1) may invite explosion due to the peroxy acid used in the reaction, the process (2) requires special facilities for electrolysis, and the process (3) requires the use of an expensive oxidizing agent in an amount of two equivalents or more and cannot avoid waste of a heavy metal. Thus, these processes are not suitable for commercial production.

The process (4) uses sodium hypochlorite, an inexpensive and safety oxidizing agent and is suitable for commercial production to some extent. However, the process is still insufficient in a low yield, the use of an excess amount of an aqueous solution of sodium hypochlorite and thus a low volume efficiency.

In the processes (5), conditions for producing arabinose from sodium gluconate using sodium hypochlorite are optimized and the arabinose yield and volume efficiency are improved as compared with the process (4). However, these processes are still insufficient for commercial production from the viewpoints of safety and operability, since the aqueous solution of sodium hypochlorite must be rapidly added under heating.

### SUMMARY OF THE INVENTION

Accordingly, it is an object of the present invention to provide a process for industrially producing an aldose having n-1 carbon atoms by decarboxylating an aldonic acid having n carbon atoms in a high yield at low cost with safety.

After intensive and detailed investigation on decarboxylation of an aldonic acid using hypochlorous acid or a hypochlorite as an oxidizing agent, the present inventors have found 1) that a reaction between the target aldose having n-1 carbon atom and the oxidizing agent (hereinafter briefly referred to “over reaction”) invites a decreased yield, and 2) that the excess reaction does not substantially proceed while the material aldonic acid remains in the reaction system.

They also have found that the over reaction can be effectively inhibited by adding a compound having reactivity with the hypochlorous acid or hypochlorite higher than that with the resulting aldose to the reaction system to thereby react with or decompose unreacted hypochlorous acid or hypochlorite that causes the over reaction. More specifically, they have found that reducing agents such as sulfites and thiosulfates, aldehydes such as formaldehyde and acetaldehyde, and carboxylic acids such as formic acid and acetic acid work as the compound having specific reactivity and thereby inhibit the over reaction.

The decarboxylation with a hypochlorite is generally performed under acidic conditions (J. Am. Chem. Soc., 81, 5190 (1959)). However, the present inventors have found 1) that an aldonic acid is in equilibrium with its corresponding lactone under acidic conditions, 2) that the lactone does not undergo decarboxylation, and 3) that the ring opening of the lactone proceeds at a rate much slower than that of decarboxylation. Accordingly, when a mixture of an aldonic acid and its corresponding lactone is subjected to decarboxylation, the over reaction of the formed aldose occurs prior to the ring opening of the lactone, thus causing a decreased yield.

They have found that the target aldose can be efficiently produced by hydrolyzing a mixture of the aldonic acid and the corresponding lactone with a base to convert all the lactone into the aldonic acid and then subjecting the reaction mixture to decarboxylation under such conditions that the lactone is not formed.

The present invention has been accomplished based on these findings. Specifically, the present invention provides a process for producing an aldose having n-1 carbon atoms from an aldonic acid having n carbon atoms with hypochlorous acid or a hypochlorite, the process including treating a reaction mixture with a compound having reactivity with the hypochlorous acid or hypochlorite higher than that with the produced aldose.

The present invention can provide an industrial process for producing an aldonic acid as a result of degradation using hypochlorous acid or a hypochlorite in a high yield at low cost with safety by substantially inhibiting the over reaction of the product.

### DESCRIPTION OF THE PREFERRED EMBODIMENTS

The process of the present invention is for producing an aldose having n-1 carbon atoms from an aldonic acid having n carbon atoms with hypochlorous acid or a hypochlorite and comprises adding a compound having reactivity with the hypochlorous acid or hypochlorite higher than that with the produced aldose to a reaction system.

Examples of the aldonic acid for use in the present invention include, but are not limited to, gluconic acid, mannonic acid, galactonic acid, gulonic acid, idonic acid, talonic acid, allonic acid, altronic acid, lyxonic acid, xylonic acid, arabinonic acid, ribonic acid, threonic acid, erythronic acid, 3-deoxygluconic acid, 3-deoxymannonic acid, 3-deoxygalactonic acid, 3-deoxygulonic acid, 3-deoxyidonic acid, 3-deoxytalonic acid, 3-deoxyallonic acid, 3-deoxyaltronic acid, 3-deoxylyxonic acid, 3-deoxyxylonic acid, 3-deoxyarabinonic acid, 3-deoxyribonic acid, 3-deoxythreonic acid, 3-deoxyerythronic acid, and salts of these acids. Any of D-aldonic acids and L-aldonic acids can be used in the decarboxylation in the present invention. Examples of salts of the aldonic acid are salts of alkali metals such as sodium, potassium and lithium; salts of alkaline earth metals such as magnesium, calcium and barium; salts of metals such as aluminum and iron; ammonium salts; and salts of primary, secondary or tertiary alkylamines.

Examples of the primary amines are alkylamines such as methylamine, ethylamine, propylamine, isopropylamine, butylamine and hexylamine; cycloalkylamines such as cyclohexylamine; and benzylamine.

Examples of the secondary amines are dialkylamines such as diethylamine, diisopropylamine, dibutylamine and dihexylamine; dicycloalkylamines such as dicyclohexylamine; and cyclic amines such as piperidine, morpholine and N-methylpiperazine.

Examples of the tertiary amines are tertiary alkylamines such as trimethylamine, triethylamine, tripropylamine, tributylamine, N-methylpiperidine and N-methylmorpholine; anilines such as aniline and N,N-dimethylaniline; and pyridines such as pyridine and 2,6-dimethylpyridine.

The process of the present invention is applied, for example, to a compound represented by following Formula (1):

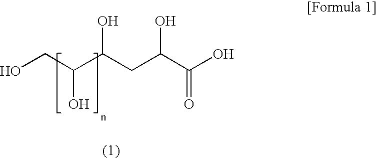

wherein n is 0 or 1, or a salt thereof.

The compound of Formula (1) or a salt thereof is not specifically limited in configuration of all the hydroxyl groups in the molecule.

The compounds of Formula (1) can be prepared, for example, by a method described in Acta. Chem. Scand., B35, 155 (1981); Carbohydr. Res. 36, 185 (1974); and Carbohydr. Res., 115, 288 (1983).

Examples of the degraded aldose produced according to the present invention include, but are not limited to, ribose, arabinose, xylose, lyxose, erythrose, threose, 2-deoxyribose, 2-deoxyxylose and 2-deoxyerythrose. The process can produce any of D-aldoses and L-aldoses.

The compound of Formula (1) yields a compound represented by following Formula (2):

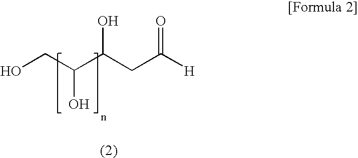

wherein n is 0 or 1.

The compound of Formula (2) is not specifically limited in configuration of all the hydroxyl groups in the molecule.

Examples of the hypochlorite for use as an oxidizing agent in the present invention are hypochlorites of an alkali metal, such as sodium hypochlorite and potassium hypochlorite; and hypochlorites of an alkaline earth metal such as calcium hypochlorite and lithium hypochlorite. The hypochlorite can be used in any form not specifically limited and can be used in the form of, for example, an aqueous solution or a solid. The hypochlorite can also be used by allowing a metal hydroxide such as sodium hydroxide to react with chloride to thereby yield a hypochlorite in the reaction system.

In general, the hypochlorite such as sodium hypochlorite is used in the form of an aqueous solution. The concentration of such an aqueous solution of the hypochlorite is not specifically limited but is, e.g., in the case of sodium hypochlorite aqueous solution, preferably 5% or more and more preferably 8% or more for better cost efficiency and volume efficiency. The upper limit thereof is not specifically limited, as long as satisfactory stability of the reagent and/or safety in operation is obtained.

The amount of the hypochlorous acid or hypochlorite is not specifically limited and is, for example, from 0.8 to 3.0 equivalents, preferably from 1.0 to 1.7 equivalents, and more preferably from 1.1 to 1.5 equivalents to the reaction substrate aldonic acid.

The hypochlorous acid or hypochlorite can be added at any rate not specifically limited and is preferably added dropwise while controlling the reaction rate and reaction temperature for satisfactory operation and safety.

The decarboxylation is generally performed in the presence of a solvent. The solvent is not specifically limited, as long as the reaction proceeds, and is preferably water or another solvent that can dissolve the raw material therein.

The reaction temperature of the decarboxylation is generally equal to or higher than 0° C. and equal to or lower than the boiling point of the solvent, preferably from 10° C. to 50° C., and more preferably from 20° C. to 50° C.

The concentration of the reaction substrate aldonic acid is not specifically limited and is preferably 10% or more, more preferably 20% or more and further preferably 30% or more at the time when the aldonic acid is added to the reaction system, for satisfactory cost efficiency and volume efficiency. The upper limit of the concentration of the reaction substrate is not specifically limited, as long as satisfactory solubility of the substrate, operability and safety are obtained.

The decarboxylation can generally be performed under acidic conditions, for example, at pH 3.5 to 6. For inhibiting lactonization, it is generally performed at pH 4 or more, preferably pH 4 to 6 and more preferably pH 4.5 to 6.

When a basic hypochlorite such as an aqueous solution of sodium hypochlorite is used, the hypochlorite is preferably added to the reaction system simultaneously with an acid for controlling pH.

Examples of the acid are a mineral acid and a carboxylic acid. As the mineral acid, hydrochloric acid is preferred. As the carboxylic acid, formic acid and acetic acid are preferred.

A mixture of the aldonic acid and its corresponding lactone may be subjected to the decarboxylation as intact, but is preferably subjected to hydrolysis with a base to hydrolyze the lactone and is then subjected to decarboxylation under such conditions that the lactone is not formed by using a pH adjuster.

The reaction temperature of hydrolysis is not specifically limited, as long as the reaction proceeds, and is generally equal to or higher than 0° C. and equal to or lower than the boiling point of the solvent, and preferably from 20° C. to 60° C.

The base for use in the hydrolysis is not specifically limited but is preferably sodium hydroxide or sodium carbonate.

The amount of the base is not specifically limited, as long as the lactone does no more remain in the reaction system.

The conditions under which the lactone is not formed are such that the pH is 4 or more, preferably from 4 to 6, and more preferably from 4.5 to 6.0.

An acid, preferably hydrochloric acid, is used as the pH adjuster after hydrolysis to adjust the pH within the above range.

The over reaction of the degraded aldose in the reaction mixture can be inhibited by adding a compound having reactivity with the hypochlorous acid or hypochlorite higher than that with the aldose to the reaction system.

The compound having the specific reactivity is not specifically limited, as long as it does not react with the aldonic acid and the aldose, and can be at least one selected from reducing agents, aldehydes and carboxylic acids.

Examples of the reducing agents are sulfites of an alkali metal, such as sodium sulfite, sodium hydrogen sulfite and sodium pyrosulfite; and thiosulfates of an alkali metal, such as sodium thiosulfate.

Among them, sodium sulfite and sodium thiosulfate are preferable.

Examples of the aldehydes are aliphatic aldehydes and aromatic aldehydes. Preferred aliphatic aldehydes are those each having one to four carbon atoms, such as formaldehyde, acetaldehyde, propionaldehyde and butyraldehyde, of which formaldehyde and acetaldehyde are more preferred. The aromatic aldehydes include, for example, benzaldehyde.

Examples of the carboxylic acids are aliphatic carboxylic acids and aromatic carboxylic acids.

Examples of the aliphatic carboxylic acids are aliphatic carboxylic acids each having one to four carbon atoms, such as formic acid, acetic acid, propionic acid, butyric acid, pivalic acid and lactic acid; as well as valeric acid.

The aromatic carboxylic acids include, for example, benzoic acid.

Among the carboxylic acids, aliphatic carboxylic acids are preferred, of which formic acid and acetic acid are more preferred.

The amount of the compound having the specific reactivity is not specifically limited, as long as the hypochlorous acid or hypochlorite in the reaction system reacts with or decomposes by action of the compound.

The hypochlorous acid or hypochlorite in the reaction system can be detected according to a conventional procedure, such as a process of using an aqueous solution of potassium iodide or measurement of oxidation-reduction potential (Courses in Experimental Chemistry, 3rd Ed., vol. 9-1, pp. 246-249).

The compound having the specific reactivity can be added to the reaction system without extra treatment such as pH adjustment.

In investigations for inhibiting the excess reaction of the aldose, the present inventors have found the difference in reactivity with the hypochlorous acid or hypochlorite in the reaction system among the aldonic acid, reducing agent, aldehyde, carboxylic acid and aldose and have found a suitable addition procedure of the compound.

If a reducing agent is used as the compound, the reducing agent has reactivity with the hypochlorous acid or hypochlorite higher than that of the material aldonic acid and is thereby preferably added at the time when the aldonic acid is consumed. In general, the reducing agent is added to the reaction system after the completion of addition of the hypochlorous acid or hypochlorite.

If an aldehyde is used as the compound, the aldehyde has reactivity with the hypochlorous acid or hypochlorite lower than that of the material aldonic acid and can be added to the reaction system at any time before, during or after addition of the hypochlorous acid or hypochlorite.

If a carboxylic acid is used as the compound, the carboxylic acid has reactivity with the hypochlorous acid or hypochlorite lower than that of the material aldonic acid and can be added to the reaction system during or after addition of the hypochlorous acid or hypochlorite.

The carboxylic acid can also be added before addition of the hypochlorous acid or hypochlorite. In this case, it should preferably be added so that pH falls within the above-specified range (generally pH 4 or higher). The carboxylic acid can work both to inhibit the excess reaction and to adjust pH in the decarboxylation. In this case, it is added simultaneously with the addition of the hypochlorous acid or hypochlorite.

Two or more of the reducing agents, aldehydes and carboxylic acids can be used in combination as the compound having the specific reactivity. In this case, these compounds can be added according to the aforementioned procedures. For example, it is preferred that an aldehyde is added before the addition of the hypochlorous acid or hypochlorite, and then acetic acid is added to the reaction system simultaneously with the hypochlorous acid or hypochlorite.

### EXAMPLES

The present invention will be illustrated in further detail with reference to several examples and comparative examples below, which are not intended to limit the scope of the invention.

### Example 1

Production of D-2-deoxyribose with a reducing agent

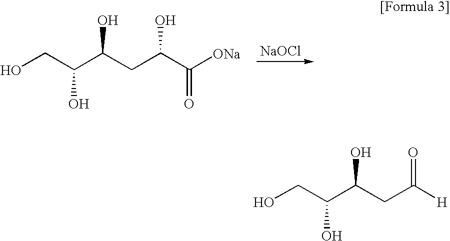

To a 25 percent by weight aqueous solution (44.7 g) of sodium 3-deoxy-D-mannonate under water-cooling was added dropwise a 8.9 percent by weight aqueous solution (60.2 g) of sodium hypochlorite over 1 hour, while controlling pH within a range from 4.5 to 5.0. Immediately after the completion of dropwise addition, sodium sulfite was added. In this procedure, it was checked using an aqueous solution of potassium iodide that hypochlorous acid was not detected. The reaction system was sequentially analyzed by high-performance liquid chromatography (HPLC), and the result is shown in Table 1. Table 1 shows that D-2-deoxyribose was produced in a reaction yield of 95% with substantially no excess reaction.

Conditions for HPLC analyses of the material and product are as follows.

Conditions for HPLC analysis of the material:

Column: Shodex Asahipak NH2P-50 4E (from Showa Denko K. K.)

Flow rate: 1 ml/min.

Column temperature: 40° C.

Detection wavelength: 210 nm

Mobile phase: 50 mM NaH₂PO₄ aqueous solution Conditions for HPLC analysis of the product:

Column: Shodex Sugar SC1011(from Showa Denko K. K.)

Flow rate: 1 ml/min.

Column temperature: 80° C.

Detector: light scattering detector (detection sensitivity: 400 mV, Ti: 35° C., T2: 45° C., nitrogen pressure: 0.15 MPa)

Mobile phase: H₂O

TABLE 1
Sequential Analysis of the Reaction

| | Time (hour) | | |
|---|---|---|---|
| | 0 | 1 | 4 |
| Yield (%) | 0 | 93 | 95 |
| Residual percentage of material (%) | 100 | 4 | 4 |
| Total (%) | 100 | 97 | 99 |

### Example 2

Production of D-2-deoxyribose by hydrolysis of a lactone with a base and decarboxylation with a reducing agent

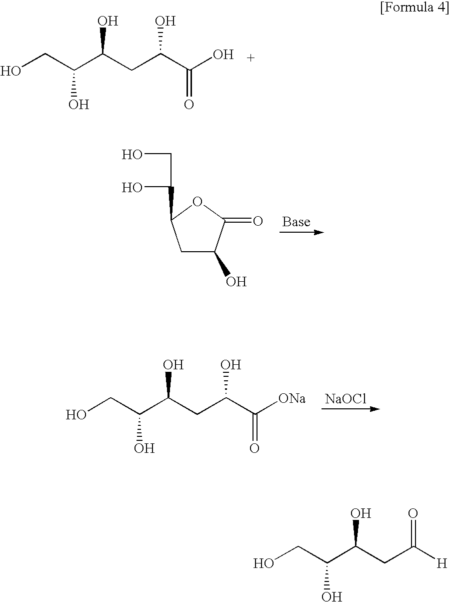

To a 28 percent by weight (in terms of carboxylic acids) aqueous solution (13.0 g) of a 86:14 by mole mixture of 3-deoxy-D-mannonic acid and 3-deoxy-D-mannonolactone was added sodium carbonate at room temperature to pH 8.8 to thereby hydrolyze the lactone. A 8.9 percent by weight aqueous solution (21.3 g; 1.26 equivalent) of sodium hypochlorite was added dropwise to the reaction mixture over 1 hour under water-cooling while adjusting the pH within a range from 4.5 to 5.0 with concentrated hydrochloric acid. Immediately after the completion of dropwise addition, sodium sulfite was added. In this procedure, it was checked using an aqueous solution of potassium iodide that hypochlorous acid was not detected. The reaction system was sequentially analyzed by HPLC, and the result is shown in Table 2.

Table 2 shows that D-2-deoxyribose was produced from a mixture with corresponding lactone in a reaction yield of 93% with substantially no excess reaction.

TABLE 2
Sequential Analysis of the Reaction

Time (hour)

| | 0 | 1 | 2.5 | 4 |
|---|---|---|---|---|
| Yield (%) | 0 | 93 | 93 | 93 |
| Residual percentage of | 100 | 7 | 7 | 7 |
| material (%) | | | | |
| Total (%) | 100 | 100 | 100 | 100 |

### Example 3

Lactonization conditions of sodium 3-deoxy-D-mannonate

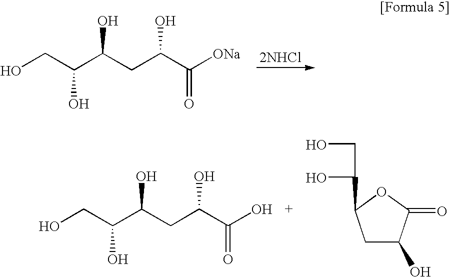

Using a 20 percent by weight aqueous solution of sodium 3-deoxy-D-mannonate, the ratio of 3-deoxy-D-mannonic acid to its corresponding lactone was determined by HPLC at different pH which was adjusted using a 2 N hydrochloric acid.

Table 3 shows that the corresponding lactone is not substantially formed at pH 4 or higher.

TABLE 3
Ratio of 3-deoxy-D-mannonic acid to its
corresponding lactone at different pH

| | 3-Deoxy-D-mannonic acid | Lactone |
|---|---|---|
| pH 2 | 85 | 15 |
| pH 3 | 95 | 5 |
| pH 4 | 98 | 2 |
| pH 4.5 | 99 | 1 |
| pH 5 | 100 | 0 |
| pH 6 | 100 | 0 |

### Example 4

Production of 2-deoxy-N-phenyl-D-ribosylamine with a reducing agent

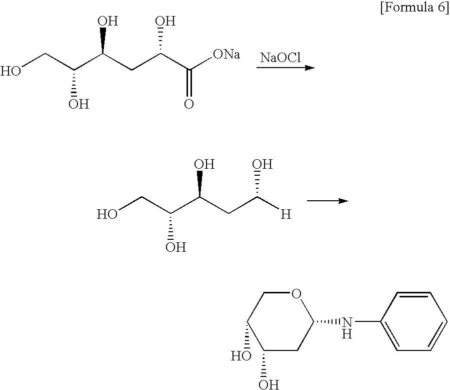

To a 24 percent by weight aqueous solution (37.2 g) of sodium 3-deoxy-D-mannonate under water-cooling was added dropwise a 12.2 percent by weight aqueous solution (39.5 g) of sodium hypochlorite over 1 hour, while controlling pH within a range of 4.5 to 5.0 with concentrated hydrochloric acid. Sodium thiosulfate as an aqueous solution was added immediately after the completion of dropwise addition. After checking, using an aqueous solution of potassium iodide, that hypochlorous acid was not detected, the reaction mixture was neutralized with sodium hydrogen carbonate.

Resulting D-2-deoxyribose was allowed to react with 1.4 g of aniline to yield 2-deoxy-N-phenyl-D-ribosylamine, collected by filtration and isolated in a yield of 80% on the basis of sodium 3-deoxy-D-mannonate.

The analysis data are shown below.

¹H-NMR (DMSO): 1.7-1.9 (2H, m), 3.4-3.7 (4H, m), 4.39 (1H, d), 4.6-4.7 (2H, m), 6.38 (1H, d), 6.5-6.7 (3H, m), 7.0-7.1 (2H, m)

### Example 5

Production of D-2-deoxyribose

After adjusting a 36.9 percent by weight aqueous solution (40 g) of sodium 3-deoxy-D-mannonate to pH 5 with hydrochloric acid, a 13 percent by weight aqueous solution (36.5 g) of sodium hypochlorite was added dropwise over 1 hour, while keeping the reaction temperature within a range from 40° C. to 45° C. and controlling pH within a range from 5 to 6 with acetic acid. After the completion of dropwise addition, the reaction mixture was aged for 1 hour and thereby yielded an aqueous solution (80.2 g) containing 9.3 g of target D-2-deoxyribose in a yield of 95% on the basis of 3-deoxy-D-mannonic acid. The oxidation-reduction potential in the reaction was 370 mV before the addition of sodium hypochlorite, was 960 to 1030 mV during the addition and dropped to 540 mV 30 minutes after the completion of the addition.

### Example 6

Production of D-2-deoxyribose

After adjusting a 36.9 percent by weight aqueous solution (40 g) of sodium 3-deoxy-D-mannonate to pH 5 with hydrochloric acid, a 13 percent by weight aqueous solution (59 g) of sodium hypochlorite was added dropwise over 1 hour, while keeping the reaction temperature within a range from 40° C. to 45° C. and controlling pH within a range from 5 to 6 with formic acid. After the completion of dropwise addition, the reaction mixture was aged for 1 hour and thereby yielded an aqueous solution (100.2 g) containing 9.1 g of target D-2-deoxyribose in a yield of 93% on the basis of 3-deoxy-D-mannonic acid. The oxidation-reduction potential in the reaction was 370 mV before the addition of sodium hypochlorite, was 960 to 1030 mV during the addition and dropped to 510 mV 30 minutes after the completion of the addition.

### Example 7

Production of D-2-deoxyribose

After adjusting a 36.9 percent by weight aqueous solution (40 g) of sodium 3-deoxy-D-mannonate to pH 5 with hydrochloric acid, a 40% aqueous solution (1.3 g) of formaldehyde was added. Then, a 13 percent by weight aqueous solution (36.5 g) of sodium hypochlorite was added dropwise over 1 hour, while keeping the reaction temperature within a range from 40° C. to 45° C. and controlling pH within a range from 5 to 6 with hydrochloric acid. After the completion of dropwise addition, the reaction mixture was aged for 1 hour and thereby yielded an aqueous solution (83.3 g) containing 9.2 g of target D-2-deoxyribose in a yield of 94% on the basis of 3-deoxy-D-mannonic acid. The oxidation-reduction potential in the reaction was 370 mV before the addition of sodium hypochlorite, was 960 to 1030 mV during the addition and dropped to 530 mV 30 minutes after the completion of the addition.

### Example 8

Production of D-2-deoxyribose

After adjusting a 36.9 percent by weight aqueous solution (40 g) of sodium 3-deoxy-D-mannonate to pH 5 with hydrochloric acid, a 90 percent by weight aqueous solution (0.8 g) of acetaldehyde was added. Then, a 13 percent by weight aqueous solution (36.5 g) of sodium hypochlorite was added dropwise over 1 hour, while keeping the reaction temperature within a range from 40° C. to 45° C. and controlling pH within a range from 5 to 6 with hydrochloric acid. After the completion of dropwise addition, the reaction mixture was aged for 1 hour and thereby yielded an aqueous solution (82.8 g) containing 9.2 g of target D-2-deoxyribose in a yield of 94% on the basis of 3-deoxy-D-mannonic acid. The oxidation-reduction potential in the reaction was 370 mV before the addition of sodium hypochlorite, was 960 to 1030 mV during the addition and dropped to 530 mV 30 minutes after the completion of the addition.

### Example 9

Production of D-2-deoxyribose

After adjusting a 36.9 percent by weight aqueous solution (40 g) of sodium 3-deoxy-D-mannonate to pH 5 with hydrochloric acid, a 90% aqueous solution (0.8 g) of acetaldehyde was added. Then, a 13 percent by weight aqueous solution (36.5 g) of sodium hypochlorite was added dropwise over 1 hour, while keeping the reaction temperature in a range from 40° C. to 45° C. and controlling pH within a range from 5 to 6 with acetic acid. After the completion of dropwise addition, the reaction mixture was aged for 1 hour and thereby yielded an aqueous solution (81 g) containing 9.2 g of target D-2-deoxyribose in a yield of 94% on the basis of 3-deoxy-D-mannonic acid. The oxidation-reduction potential in the reaction was 370 mV before the addition of sodium hypochlorite, was 960 to 1030 mV during the addition and dropped to 550 mV 30 minutes after the completion of the addition.

### Comparative Example 1

Production of D-2-deoxyribose

The procedure of Example 5 was repeated, except for using hydrochloric acid instead of acetic acid, and the oxidation-reduction potential was determined. The oxidation-reduction potential in the reaction was 370 mV before the addition of sodium hypochlorite, was 960 to 1070 mV during the addition, but dropped only to 860 mV 30 minutes after the completion of the addition and dropped to 570 mV 1.5 hours after the completion of the addition.

### Example 10

Production of D-2-deoxythreose

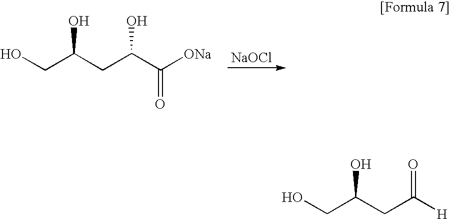

To a 11.4 percent by weight aqueous solution (44.5 g) of sodium 3-deoxy-D-lyxonate was added dropwise a 11.7 percent by weight aqueous solution (24.4 g) of sodium hypochlorite over 1 hour, while controlling pH within a range from 4.5 to 5.0 with concentrated hydrochloric acid under water-cooling. An aqueous solution of sodium thiosulfate was added immediately after the completion of dropwise addition. The reaction mixture was adjusted to pH 7.3 with a 2 N aqueous solution of sodium hydroxide, and water was evaporated and removed at 50° C. The residue was mixed with 10 ml of methanol, and the precipitated inorganic salt was removed by filtration. The filtrate was concentrated under reduced pressure. The residue was mixed with 10 ml of methanol and 10 ml of chloroform, the precipitated inorganic salt was removed by filtration, and the filtrate was concentrated under reduced pressure. The residue was purified by silica gel chromatography (eluent: chloroform/methanol=10/1) to yield 2.4 g of D-2-deoxythreose in a yield of 78% as a colorless transparent syrup.

The data of ¹³C— and ¹H-NMR analyses of the above-prepared compound (Table 4) agree with the literature values [Carbohydrate Research, 210, 21 (1991); and Tetrahedron Asymmetry, 9, 1359 (1998)].

D-2-deoxythreose is present as a mixture in equilibrium of α-anomer, β-anomer and a corresponding hydrate in D₂O.

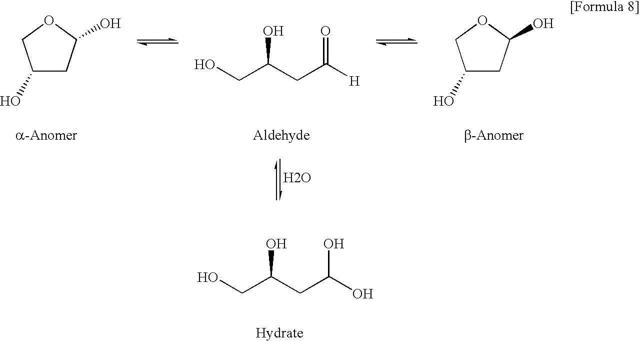

TABLE 4
¹³C-NMR (D₂O)
Reference material: DSS [sodium 3-(trimethylsilyl)-1-
propanesulfonate]

| | C-1 | C-2 | C-3 | C-4 |
|---|---|---|---|---|
| ¹³C-NMR (ppm) | position | position | position | position |
| α-Anomer (measured) | 100.8 | 43.6 | 72.5 | 76.9 |
| β-Anomer (measured) | 100.7 | 44.5 | 73.7 | 76.0 |
| Hydrate (measured) | 91.3 | 43.0 | 71.3 | 68.1 |
| α-Anomer | 99.7 | 42.6 | 71.5 | 75.7 |
| (literature) | | | | |
| β-Anomer | 99.7 | 43.5 | 72.6 | 75.0 |
| (literature) | | | | |
| Hydrate (literature) | 89.9 | 41.5 | 69.8 | 66.7 |

¹H-NMR (D₂O)
Chemical shift of proton at the anomeric position

| ¹H-NMR ppm | α-Anomer | β-Anomer | Hydrate |
|---|---|---|---|
| Measured value | 5.49 | 5.65 | 5.18 |
| Literature value | 5.49 | 5.65 | 5.18 |

### Example 11

Production of D-2-deoxythreose

After adjusting a 25 percent by weight aqueous solution (20.2 g) of sodium 3-deoxy-D-lyxonate to pH 5 with hydrochloric acid, a 11.6 percent by weight aqueous solution (29.9 g) of sodium hypochlorite was added dropwise over half an hour, while keeping the reaction temperature within a range from 40° C. to 45° C. and controlling pH within a range from 5 to 6 with acetic acid. After aging for 1 hour, the reaction mixture was adjusted to pH 7.3 with a 2 N aqueous solution of sodium hydroxide, and water was evaporated and removed at 50° C. The residue was mixed with 10 ml of methanol, and the precipitated inorganic salt was removed by filtration. The filtrate was concentrated under reduced pressure. The residue was mixed with 10 ml of methanol and 10 ml of chloroform, the precipitated inorganic salt was removed by filtration, and the filtrate was concentrated under reduced pressure. The residue was purified by silica gel chromatography (eluent: chloroform/methanol=10/1) to yield 2.5 g of D-2-deoxythreose in a yield of 81% as a colorless transparent syrup.

What is claimed is:

1. A process for producing an aldose having n-1 carbon atoms from an aldonic acid having n carbon atoms with hypochlorous acid or a hypochlorite, the process comprising treating a reaction mixture with a compound having reactivity with the hypochlorous acid or hypochlorite higher than that of the produced aldose.

2. The process according to claim 1, wherein the compound is at least one selected from the group consisting of a reducing agent, an aldehyde and a carboxylic acid.

3. The process according to claim 2, wherein the reducing agent is at least one selected from alkali metal sulfites and alkali metal thiosulfates, wherein the aldehyde is at least one selected from aliphatic aldehydes having one to four carbon atoms and aromatic aldehydes, and wherein the carboxylic acid is at least one selected from aliphatic carboxylic acids having one to four carbon atoms and aromatic carboxylic acids.

4. The process according to claim 3, wherein the compound is a reducing agent which is at least one of sodium sulfite and sodium thiosulfate.

5. The process according to claim 3, wherein the compound is an aldehyde which is at least one of formaldehyde and acetaldehyde.

6. The process according to claim 3, wherein the compound is a carboxylic acid which is at least one of formic acid and acetic acid.

7. The process according to claim 1, further comprising subjecting a mixture of the aldonic acid and its corresponding lactone to hydrolysis with a base and then subjecting the hydrolyzed product to decarboxylation under such conditions that the lactone is not formed.

8. The process according to claim 1, wherein the aldonic acid is a compound represented by following Formula (I) or a salt thereof: 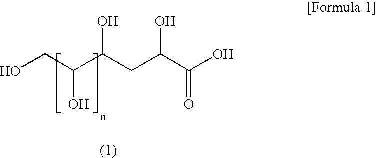
wherein n is 0 or 1, and wherein the aldose is a compound represented by following Formula (2): 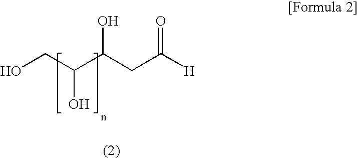
wherein n is 0 or 1.
